(19) United States
(12) Patent Application Publication
(10) Pub. No.: US 2026/0350803 A1
(43) Pub. Date: Nov. 5, 2026

(54) USE OF SUPERELLIPSE SETS FOR MYOCARDIAL IMAGING

(71) Applicant: Siemens Medical Solutions USA, Inc., Malvern, PA (US)

(72) Inventors: Maximilian Reymann, Erlangen (DE); Alexander Hans Vija, Evanston, IL (US)

(21) Appl. No.: 19/558,595

(22) Filed: Mar. 6, 2026

Related U.S. Application Data
(60) Provisional application No. 63/798,727, filed on May 2, 2025.

(51) Int. Cl. G06T 7/00; A61B 5/055; A61B 6/00; A61B 6/03; A61B 6/50; G06T 5/60; G06T 5/73; G06T 7/246; G06T 7/60

(52) U.S. Cl. CPC G06T 7/0014; A61B 5/055; A61B 6/032; A61B 6/037; A61B 6/503; A61B 6/5205; G06T 5/60; G06T 5/73; G06T 7/246; G06T 7/60; G06T 2200/04; G06T 2207/20084; G06T 2207/30048; G06T 2207/30168

(57) ABSTRACT

A system and method include determination of a first three-dimensional image, for each of a plurality of first slices of the first three-dimensional image, fitting of a model comprising a plurality of parameters to a first object in the first slice to determine values of each of the plurality of parameters, determination of values of each of the plurality of parameters for each of a plurality of second slices of a second three-dimensional image of a second object, determination of a difference between the values of each of the plurality of parameters determined for each of the plurality of first slices and the values of each of the plurality of parameters determined for each of the plurality of second slices, and outputting of an indication based on the difference.

### CROSS-REFERENCE TO RELATED APPLICATIONS

This application claims priority to, and the benefit of, U.S. Provisional Patent Application Ser. No. 63/798,727, filed May 2, 2025, the contents of which are incorporated herein by reference for all purposes.

### BACKGROUND

Modern medical imaging systems (e.g., echocardiography, computed tomography (CT), single-photon emission computed tomography (SPECT), positron emission tomography (PET) and magnetic resonance imaging (MRI)) are capable of acquiring detailed anatomical images. These images may be used for diagnosis and treatment planning. The quality of diagnosis and treatment planning depends on the quality of the images.

Image quality can be affected by inadequate system calibration, modality-specific imaging characteristics and patient motion. Image corrections are therefore applied to acquired image data to account for these effects. The quality of these corrections is conventionally assessed through qualitative reader studies, in which multiple experts manually compare images to which various corrections (including no correction) have been applied. Reader studies are time consuming, expensive and subjective.

Accordingly, there is a need for systems and methods to efficiently assess the quality of a reconstructed image. Such systems and methods may be used to evaluate system calibration, patient motion and/or applied image corrections.

### BRIEF DESCRIPTION OF THE DRAWINGS

The foregoing and other aspects of some embodiments are best understood from the following detailed description when read in connection with the accompanying drawings. Embodiments are not limited to the specific implementations disclosed herein.

FIG. 1 is a block diagram of a system to characterize the shape of an anatomical structure using superellipse parameters according to some embodiments;

FIGS. 2A and 2B comprise a flow diagram of a process to characterize the shape of an anatomical structure using superellipse parameters according to some embodiments;

FIG. 3 is a slice image of a portion of a left ventricle according to some embodiments;

FIG. 4 illustrates a line through a center of the portion of the left ventricle of the FIG. 3 image according to some embodiments;

FIG. 5 is a profile of pixel intensities along the line of FIG. 4 according to some embodiments;

FIG. 6A illustrates a Gaussian function fit to a first peak of the FIG. 5 intensity profile according to some embodiments;

FIG. 6B illustrates a Gaussian function fit to a second peak of the FIG. 5 intensity profile according to some embodiments;

FIG. 7 illustrates locations of peak intensities along the line of FIG. 4 according to some embodiments;

FIG. 8 illustrates locations of peak intensities along several lines through the center of the portion of the left ventricle of the FIG. 3 image according to some embodiments;

FIG. 9 depicts a system to determine superellipse parameter values based on the peak intensities of FIG. 8 using a neural network according to some embodiments;

FIG. 10 depicts a system to characterize a region based on superellipse parameter values determined from a plurality of slice images according to some embodiments;

FIGS. 11 through 13 depict calibration of an imaging system based on superellipse parameter values determined from a plurality of slice images according to some embodiments;

FIG. 14 depicts a system to evaluate patient motion based on superellipse parameter values determined from a plurality of slice images according to some embodiments;

FIG. 15 depicts a system to evaluate different reconstruction methods based on superellipse parameter values determined from a plurality of slice images according to some embodiments;

FIG. 16 depicts conversion of sets of superellipse parameter values to idealized sets of superellipse parameter values according to some embodiments;

FIG. 17 depicts determination of a metric based on differences between sets of superellipse parameter values and corresponding idealized sets of superellipse parameter values according to some embodiments; and

FIG. 18 is a block diagram of an imaging system according to some embodiments.

### DETAILED DESCRIPTION

The following description is provided to enable any person in the art to make and use the described embodiments. Various modifications, however, will be readily-apparent to those in the art. Independent of the grammatical term usage, individuals with male, female or other gender identities are included within the term.

The present inventors have discovered a system to efficiently detect the left ventricle within an image and to quantify the shape of the left ventricle using a relatively small set of parameter values. The present inventors have further discovered that these parameter values may act as an efficient proxy for the accuracy of an image and the presence or absence of artifacts in the image. Unlike traditional qualitative methods, embodiments provide objective metrics for evaluating causes of image inaccuracy and/or artifacts, including but not limited to system mis-calibration, patient motion, and/or sub-optimal motion correction.

For instance, respiratory motion can result in artifacts that distort the shape and intensity distribution of the left ventricle in an image, impacting quantification and introducing image artifacts. A set of parameter values derived from the image can be compared to an idealized set of parameter values or to a set of parameter values derived from an optimal heart template by determining the difference in respective bounded volumes. In another example, the tracer uptake activity [kBq/ml] of the left ventricle at the positions defined by the set of parameter values can be used to compute a measure of homogeneity. The degree of homogeneity is inversely correlated to respiratory motion, which is assumed to cause local fluctuations in left ventricle uptake activity. The above measures allow determination of whether patient motion exceeded a threshold level, evaluation of different motion correction algorithms, and/or optimization of image reconstruction with the goal of minimizing the determined measures.

Optimizing image reconstruction may include determining an optimal number of gates of a respiratory- or cardiac-gated reconstruction based on derived parameter values. This determination may be performed by any system, including a trained neural network. For clinical and research applications, shapes defined by patient-specific image-derived parameter values can potentially indicate underlying pathologies, support clinical decision-making and track treatment efficacy.

Briefly, some embodiments determine a first three-dimensional image and, for each of a plurality of first slices of the first three-dimensional image, fit a model comprising a plurality of parameters to a first object in the first slice to determine values of each of the plurality of parameters. A difference is determined between the values of each of the plurality of parameters determined for each of the plurality of first slices and values of each of the plurality of parameters determined for each of a plurality of second slices of a second three-dimensional image of a second object. An indication is output based on the difference.

In some embodiments, the determined difference is greater than a threshold and the indication includes instructions to perform a device calibration. In other embodiments, the indication is an indication that motion artifacts of the first three-dimensional image exceed a threshold. In still other embodiments, the indication is an indication of which of the first three-dimensional image and the second three-dimensional image exhibits a higher image quality. Each of the above aspects will be discussed in detail below, and embodiments are not limited to those aspects.

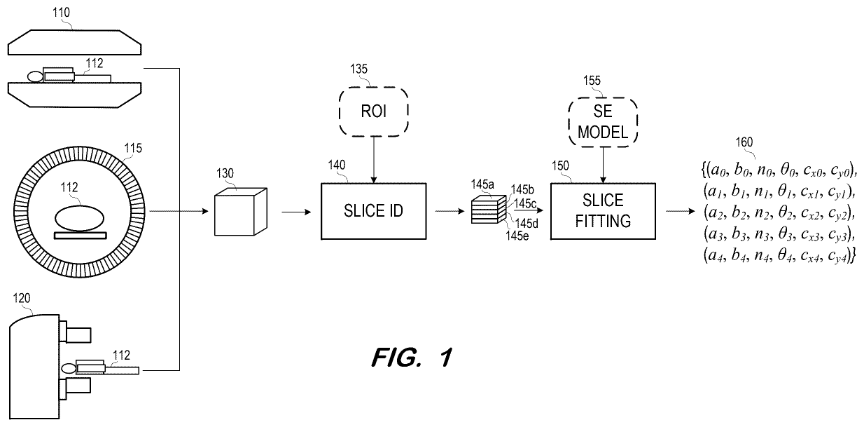

FIG. 1 is a block diagram of a system to characterize the shape of an object using superellipse parameters according to some embodiments. Each component depicted in FIG. 1 may be implemented using any combination of hardware and/or software (i.e., executable program code), and one or more components may be implemented using the same hardware and/or software.

The object, e.g., an anatomical structure, is depicted in three-dimensional image 130, which is a three-dimensional set of pixel intensities representing materials within a volume. Image 130 may be acquired/generated using any system that is or becomes known. As non-exhaustive examples, FIG. 1 indicates that image 130 may be an image of a volume of patient 112 which was generated using MR scanner 110, PET scanner 115 or SPECT scanner 120. In the case of scanners 115 and 120, image 130 may be generated by acquiring data and reconstructing the data based on a linear attenuation coefficient map of patient 112 acquired by, e.g., a CT scanner. Image 130 may undergo any suitable processing or corrections that are or become known, including but not limited to motion correction.

Region of interest 135 of image 130 is identified. For example, an operator may manipulate a user interface displaying image 130 to indicate a volume of interest which includes the object. An automated or semi-automated segmentation algorithm may be executed to identify a predetermined region of interest 135. In the foregoing description, region of interest 135 includes a left ventricle depicted in image 130.

Slice identifier 140 may determine a three-dimensional sub-portion of image 130 which includes region of interest 135. In some cases, for example if region of interest 135 makes up a significant portion of image 130, slice identifier 140 may operate on entire image 130. Slice identifier 140 determines slices 145a-145e of the sub-portion of image 130.

Each of slices 145a-145e may be one voxel thick, but embodiments are not limited thereto. Embodiments are also not limited to five slices. A voxel according to some embodiments is a cubic region measuring 5 mm×5 mm×5 mm. Slices 145a-145e may be oriented such that the portion of the object within each slice may be efficiently characterized by a superellipse. In a case that the region of interest is the left ventricle, slices 145a-145e may be oriented perpendicular to a major axis of the left ventricle.

Slice fitting component 150 fits the object depicted in each slice 145a-145e to superellipse model 155. According to some embodiments, superellipse model 155 is as follows:

$${f}_{i} ( x , y ) = {\vert \frac{{x}_{i}^{′}}{{a}_{i}} \vert}^{{n}_{i}} + {\vert \frac{{y}_{i}^{′}}{{b}_{i}} \vert}^{{n}_{i}} - 1 , with$$

$${x}_{i}^{′} = ( x - {c}_{x , i} ) cos {θ}_{i} + ( y - {c}_{y , i} ) sin {θ}_{i}$$

$${y}_{i}^{′} = - ( x - {c}_{x , i} ) sin {θ}_{i} + ( y - {c}_{y , i} ) cos {θ}_{i}$$

Accordingly, slice fitting component 150 determines values 160 for parameters a, b, n, θ, cₓ, c_{y} for each slice i. a represents a length of a semi-major axis, b represents a length of a semi-minor axis, n represents a squareness, or curvature, θ represents a rotation, and cₓ, c_{y} are coordinates of the center. Parameter values 160 advantageously provide a succinct, accurate and reproducible characterization of the three-dimensional anatomical structure. Several methods for determining parameter values 160 will be described below.

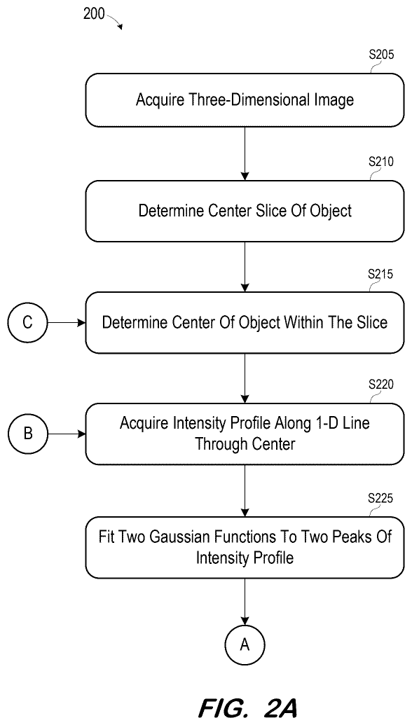

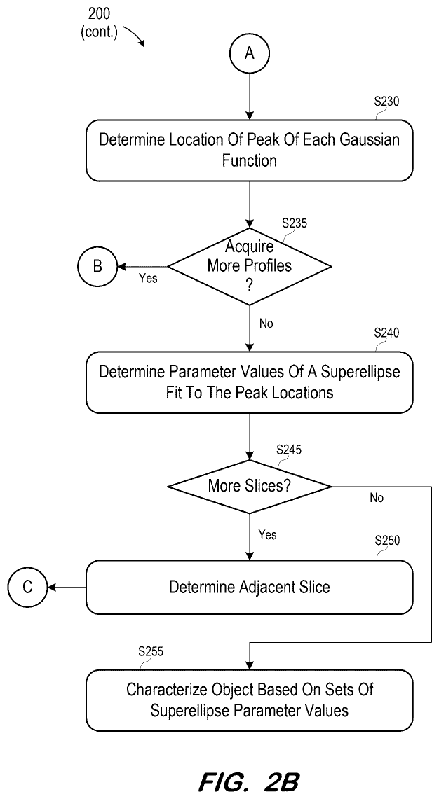

FIGS. 2A and 2B comprise a flow diagram of process 200 to characterize the shape of an object using superellipse parameters according to some embodiments. Process 200 may be performed by any combination of hardware and software that is or becomes known, and need not be performed by an imaging system. Program code embodying these processes may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processor cores, and processor threads. Embodiments are not limited to the examples described below.

Initially, a three-dimensional image is acquired at S205. The three-dimensional image may be acquired by an imaging system or from a computer-readable storage medium. For example, the three-dimensional image may have been generated days, months or years prior to S205 and in a different location. The three-dimensional image consists of voxels (which will also be referred to herein as pixels), each of which is assigned an intensity value. The intensity value at a voxel may represent a material located at the voxel as is known in the art.

A center slice of the object is determined at S210. The center point of the object (e.g., the left ventricle) may be determined from the three-dimensional image using any method or approximation that is or becomes known. The determined central slice is the slice of the image which includes the center point. As described above, the center slice may be perpendicular to the major axis of the left ventricle.

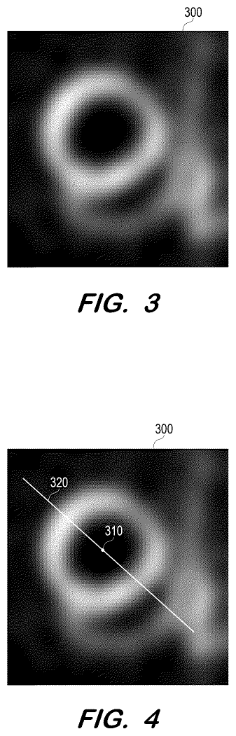

FIG. 3 shows axial slice 300 determined at S210 according to one example. The bright ring shown in slice 300 depicts a portion of the myocardial wall which resides in slice 300. A center of the object within the slice is determined at S215. In the first iteration of S215, the center of the object within the slice is the center point determined at S210.

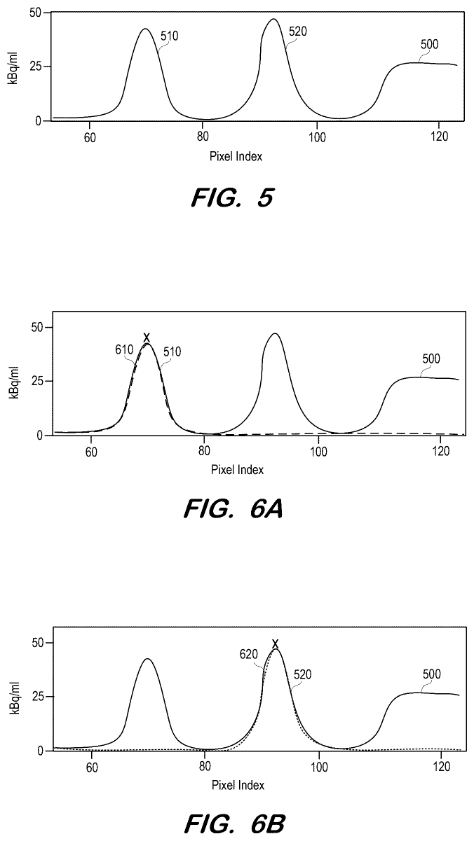

At S220, an intensity profile along a one-dimensional line through the center is acquired. FIG. 4 shows line 320 through center 310 according to some embodiments. S220 consists of determining the intensities of all pixels along line 320. FIG. 5 illustrates acquired profile 500 of the pixel intensities along line 320 according to some embodiments. As shown, profile 500 includes peaks 510 and 520 which correspond to locations of the myocardial wall within image 300.

Next, at S225, two Gaussian functions are fit to two peaks of the intensity profile. S225 may include detection of two peaks of the profile and determination of a window of intensities about each peak to which the parameters of a Gaussian function will be fitted. FIG. 6A illustrates function 610 fit to peak 510 of profile 500 according to some embodiments, and FIG. 6B illustrates function 620 fit to peak 520 of profile 500 according to some embodiments.

The intensity profile may include three or more peaks of similar magnitude, so S225 may include determining the two of the three or more peaks to which Gaussian functions will be fitted. For example, S225 may consider only peaks located within a radius of 4 cm around the center determined at S215. In some embodiments, S225 identifies the two peaks with the smallest distance to the center in order to avoid identifying additional peaks that might exist around the left ventricle.

Due to the many possible combinations of peaks and intensities of the intensity profile, some embodiments implement a robust algorithm for locating two peaks which represent the myocardial wall, locating windows about the peaks to which the Gaussian functions will be fit, and fitting the Gaussian functions. Embodiments are not limited to the foregoing algorithm.

Initially, all peaks are identified which have a minimal distance of 1 cm (assuming the diameter of the left ventricle is ≥1 cm), a peak intensity value of at least 20% of the maximum intensity value of the profile and a minimum width of one pixel. Also, all minima along the profile that have a minimal distance of 1 cm and a minimum width of 1 pixel are identified.

Next, a window of pixels is determined for each peak of the profile. Starting from the left-most identified peak, the closest minimum strictly to the left and strictly to the right (i.e., the nearest flanking minima) are identified. If no minimum exists on a side of the peak, the nearest profile boundary (i.e., pixel index 0 or the last pixel index) is identified. A symmetric window is determined which is centered on the peak and which fits entirely between the two minima which were determined for the peak. The intensity values within this symmetric window are determined and these will be the values to which a Gaussian function will be fit.

The Gaussian fit to each peak may consist of an optimization which is constrained by initialization values: amplitude=intensity value at the peak, center=pixel index at the peak, width=window width h if h<3 cm, otherwise use 3 cm. During the optimization, the amplitude may be constrained to +1% around the observed peak intensity value, the center limited to the symmetric window, and the width bounded to [half a pixel size, and 3 cm].

According to some embodiments, a weighted least squares using a Gaussian weighting is applied that emphasizes samples near the peak center (i.e., samples near the center are weighted more heavily than sample near the window edges: weight

$${r}_{i} = exp ( - \frac{{( {x}_{i} - p )}^{2}}{h / {2}^{2}} ) ,$$

where h is the current window width and p is the peak pixel index. The weighted least-squares objective minimized by the fit may be:

$$J ( A , μ , σ ) = {\sum }_{i ∈ 𝒲} {{r}_{i}^{2} [ {y}_{i} - G ( {x}_{i} ; A , μ , σ ) ]}^{2}$$

where [symbol] indexes the window samples, A is the amplitude, μ is the mean/center, σ is the gaussian width, and G is a Gaussian function.

The above process results in a Gaussian fit for all peaks found on the profile. Since the number of peaks may be greater than two, the fitted peaks are post-processed to identify two of the peaks representing the myocardial wall. For example, peaks within 0.6 cm of the profile midpoint may be excluded since they are likely caused by blood-pool activity. From the remaining peaks, the two peaks closest to the profile center are selected.

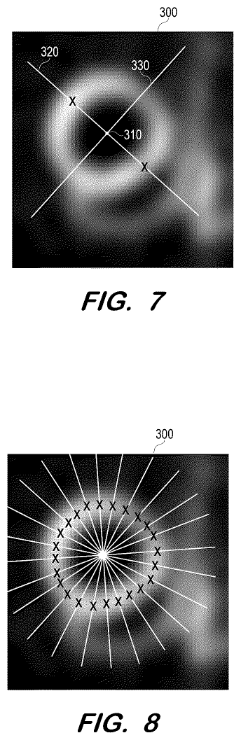

Returning to process 200, the locations of the peaks of each of the two Gaussian functions are determined at S230. Deriving the peak locations from the Gaussian functions allows sub-pixel resolution. These locations are assumed to be the (x, y) coordinates of the myocardial wall along the profile. The locations are denoted by “X” in FIGS. 6A and 6B, and corresponding locations of image 300 are denoted by “X” along line 320 of FIG. 7.

At S235 it is determined whether more intensity profiles are to be acquired. It will be assumed, for example, that twelve intensity profiles are to be acquired. Accordingly, flow returns to S220 to acquire intensity profile along another line through the center. FIG. 7 shows line 330 along which the intensity profile may be acquired.

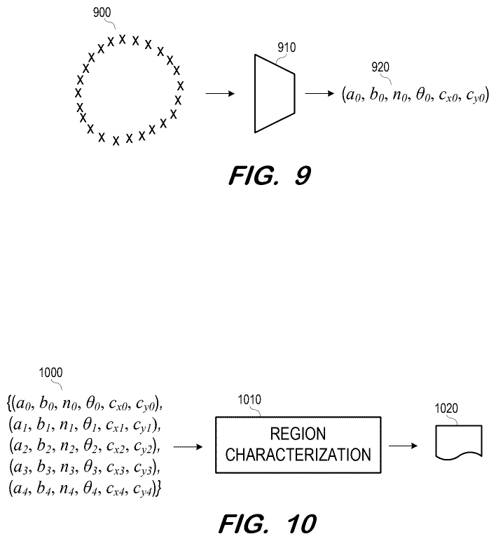

Flow cycles between S220 and S235 until two peak locations have been determined for the desired number of intensity profiles. FIG. 8 shows peak locations determined for each of twelve one-dimensional lines according to some embodiments. At S240, a superellipse is fit to the peak locations. Fitting a superellipse comprises determining values of parameters (a, b, n, θ, cₓ, c_{y}) of a superellipse based on the peak locations. S240 may include a non-linear least squares optimization regularized to physiological bounds. FIG. 9 illustrates an embodiment in which peak locations 900 are input to neural network 910 to generate superellipse parameter values 920.

At S245, it is determined whether more slices of the anatomical structure exist. If so, another slice is determined at S250 and flow returns to S215. The determined slice may be adjacent to a slice which was already processed by process 200. Using an adjacent slice allows the known location of the center for an already-processed adjacent slice to be leveraged in the determination of the center for the current slice at S215.

Flow therefore cycles between S215 and S250 to acquire a set of values of superellipse parameters for each of a plurality of slices. The sets of parameter values may constitute a comprehensive three-dimensional model of the object. The model allows interpolation along all surface points to provide a detailed geometric representation. Accordingly, once it is determined at S245 that all slices have been processed, the object may be characterized based on the sets of parameter values at S255.

FIG. 10 shows five sets 1000 of parameter values determined according to process 200. Region characterization component 1010 may receive sets 1000 and output characterization 1020. Characterization 1020 may comprise any finding which may be determined based on sets 1000. For example, characterization 1020 may comprise an indication of abnormality, i.e., a deviance from an expected “normal” shape which is greater than a threshold. In the case of the left ventricle, the fit superellipses are expected to be relatively circular (a=b and n=2). Accordingly, characterization 1020 may indicate whether the superellipses deviate significantly from this expectation.

It is known that respiratory motion manifests as elongation of the imaged left ventricle in the superior-inferior direction. Therefore, characterization 1020 may indicate excessive motion during the acquisition of the image (and/or inadequate motion correction) if the fit superellipses are not sufficiently circular. In other examples, characterization 1010 may indicate one or more of a volume of the anatomical structure, a deviance from a “normal” volume, a degree of circularity along the major axis of the anatomical structure, a shape-based diagnosis, etc.

FIGS. 11 through 13 depict calibration of an imaging system based on superellipse parameter values determined from a plurality of slice images according to some embodiments. Imaging system 1100 of FIG. 11 may comprise a SPECT/CT system but embodiments are not limited thereto. FIG. 11 includes phantom 1105 which may comprise any one or more calibration phantoms as is known or becomes known. It is assumed, for example, that imaging system 1100 has been calibrated according to corresponding quality assurance procedures which may utilize phantom 1105, one or more other phantoms, and/or other unshown devices.

Imaging system 1100 acquires image 1110 of phantom 1105. Image 1110 is intended to represent an image of phantom 1105 (or of a portion of phantom 1105 representing, for example a left ventricle) as would be acquired by a properly-calibrated system. Image 1110 is depicted as five slices as described above. Slice fitting component 1120 may operate as described above to generate, for each of the five slices of image 1110, a set of parameter values representing an object depicted within each slice. In the illustrated example, each set of parameter value sets 1130 includes a value for each of parameters a, b, n, θ, cₓ and c_{y}. Parameter value sets 1130 therefore represent a model of the three-dimensional shape of the object.

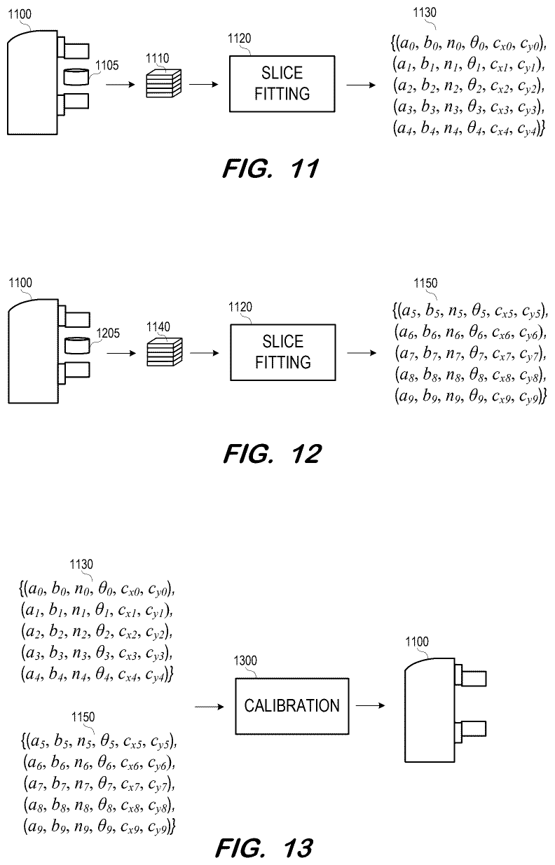

FIG. 12 depicts imaging system 1100 at a point in time after the process depicted in FIG. 11. To ensure that imaging system 1100 is still properly calibrated, imaging system acquires image 1140 of phantom 1205. Phantom 1205 may comprise a same model of phantom as phantom 1105 or may be phantom 1105. Image 1140 may comprise an image of phantom 1205 or of a portion of phantom 1205.

Slice fitting component 1120 generates parameter value sets 1150 based on slices of image 1140 as described herein. Each set of parameter value sets 1150 represents a two-dimensional shape of an object depicted in a respective slice of image 1140.

As shown in FIG. 13, calibration component 1300 may compare parameter value sets 1130 to parameter values sets 1150 to determine whether imaging system 1100 is properly calibrated. For example, significant differences in the three-dimensional shapes represented by parameter value sets 1130 and parameter values sets 1150 may indicate alignment, geometry or other calibration errors during the acquisition of image 1140. The difference may be quantified as one or more of a difference in volume circumscribed by each three-dimensional shape, a difference in major axis length or minor axis length, a difference in rotation, a difference in circularity (i.e., n), a difference in the mean value of a given parameter (e.g., ā, b̅, n̅, θ̅, c̅ₓ̅, c̅_̅{̅y̅}̅), etc.

Calibration component 1300 may output an indication to re-calibrate imaging system 1100 if the determined difference(s) is greater than a threshold(s). Component 1300 may also or alternatively output a value of one or more determined difference or other error measure, an indication of a type of calibration error suggested by the difference(s), and/or general or specific remedial actions determined based on parameter value sets 1130 and 1150.

The imaging system of FIGS. 12 and 13 may be different from imaging system 1100. For example, imaging system 1100 may be an instance of a particular product model which is used to generate calibration images, which the imaging system of FIGS. 12 and 13 may be a different instance of the product model which is being calibrated in the field.

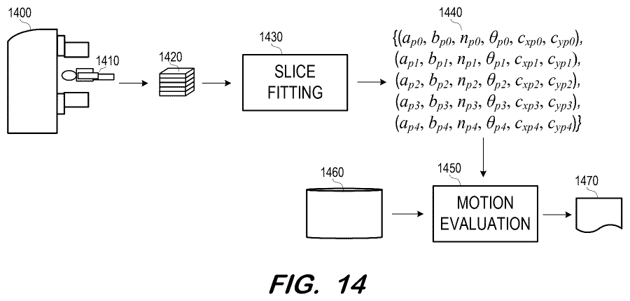

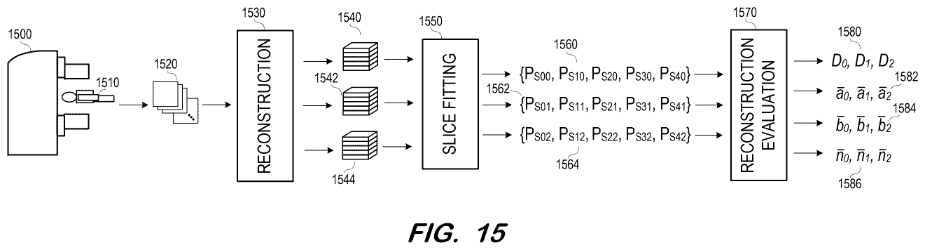

FIG. 14 depicts a system to evaluate patient motion based on superellipse parameter values determined from a plurality of slice images according to some embodiments. Imaging system 1400 may comprise a SPECT/CT system but embodiments are not limited thereto. Imaging system 1400 acquires image 1420 of patient 1410. Slice fitting component 1430 generates, for each of the five depicted slices of image 1420, a set of parameter values representing an object depicted within each slice. Taken together, sets of parameter values 1440 represent the three-dimensional shape of the object.

Sets of parameter values 1440 are input to motion evaluation component 1450 according to some embodiments. Motion evaluation component 1450 may compare sets 1440 against sets of parameter values stored in database 1460 and output evaluation 1470 indicating whether image 1420 includes excessive motion artifacts. Evaluation 1470 may indicate that image data of patient 1410 should be reacquired, that a different motion correction algorithm should be used to reconstruct image 1420, or another indication.

For example, database 1460 may include a plurality of sets of parameter values, where each one is derived from a respective image depicting an acceptable degree of object motion. The images may represent different cohorts (e.g., small bodies, large bodies, male, female). Motion evaluation component 1450 may therefore identify one or more sets of parameter values of database 1460 which are derived from images associated with a cohort of patient 1410, and compare each of the one or more sets to sets 1440. If the comparison indicates that a similarity between sets 1440 and one of the one or more sets is greater than a threshold, component 1450 may output evaluation 1470 indicating that a degree of motion (i.e., motion artifacts) present in image 1420 is acceptable.

Database 1460 may also or alternatively include a plurality of sets of parameter values in which each one is derived from an image depicting an unacceptable degree of object motion. Motion evaluation component 1450 may identify one or more of these sets of parameter values, compare each of these one or more sets to sets 1440, and output evaluation 1470 indicating excessive motion if the comparison indicates a similarity between sets 1440 and one of the one or more stored sets. In some embodiments, the images from which the sets of parameter values of database 1460 are derived are reconstructed using the same reconstruction algorithm and corrections (e.g., respiratory gating, motion correction) as used to generate image 1420.

In some embodiments, motion evaluation component 1450 compares sets 1440 to a standardized, or expected version of sets 1440. For example, the object of interest (e.g., the left ventricle) may be expected to appear circular in each slice of image 1420. Motion evaluation component 1450 therefore determines a volume of the three-dimensional shape circumscribed by sets 1440 and also determines a volume of a three-dimensional shape circumscribed by a version of sets 1440 in which each parameter n is assigned a value of 2 and each parameter b_{p #}is set equal to its corresponding parameter a_{p #}. If a difference between the volumes is greater than a threshold, component 1450 determines that image 1420 exhibits excessive motion artifacts.

In another example, a separate image of a phantom (e.g., a physical phantom, the XCAT virtual phantom) is acquired and sets of superellipse parameter values are derived from slices thereof as described herein. The volume of a three-dimensional shape circumscribed by these sets is compared to the volume of the three-dimensional shape circumscribed by sets 1440. Again, if a difference between the volumes is greater than a threshold, component 1450 determines that image 1420 exhibits excessive motion artifacts.

FIG. 15 depicts a system to evaluate different reconstruction methods based on superellipse parameter values determined from a plurality of slice images according to some embodiments. Imaging system 1500 scans patient 1510 to generate projection data 1520. Embodiments are not limited to the acquisition of projection data and may include coincidence data, k-space data or any other type of raw data. Reconstruction component 1530 reconstructs images 1540, 1542 and 1544 based on data 1520. Embodiments are not limited to three images.

In some examples, each of images 1540, 1542 and 1544 is generated from data 1520 using a different reconstruction technique. The techniques used to reconstruct images 1540, 1542 and 1544 may differ in any manner. For example, reconstruction component 1530 may apply the same reconstruction algorithm to each of images 1540, 1542 and 1544, but may apply no motion correction in the generation of image 1540, a first type of motion correction in the generation of image 1542 and a third type of motion correction in the generation of image 1544.

Slice fitting component 1550 operates on slices of images 1540, 1542 and 1544 as described above. The notation Pₛₜᵢ of FIG. 15 denotes a set of superellipse parameter values ((a, b, n, θ, cₓ, c_{y}) derived from slice t of image i. Slice fitting component 1550 determines sets 1560 of parameter values for the slices of image 1540, sets 1562 of parameter values for the slices of image 1542, and sets 1564 of parameter values for the slices of image 1544.

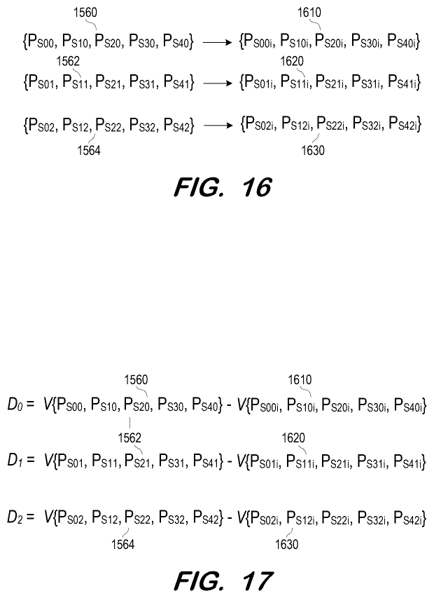

Reconstruction evaluation component 1570 may compare the quality of the reconstructions of images 1540, 1542, 1544 based on respective parameter sets 1560, 1562, 1564. A quality of an image may be determined by computing metrics which compare its associated parameter sets to their idealized versions. FIG. 16 depicts conversion of sets of superellipse parameter values to “idealized” sets of superellipse parameter values according to some embodiments. For example, sets 1560 of parameter values are converted to sets 1610 of parameter values by, as described above, changing each value of parameter n to 2 and equalizing the values of parameters a and b of each set. Similar conversions are applied to sets 1562 and 1564 to result in sets 1620 and 1630. In alternative embodiments, idealized versions 1610, 1620 and 1630 are identical and are derived from images of a corresponding XCAT phantom.

As shown in FIG. 17, depicts determination of a metric based on differences between sets of superellipse parameter values and corresponding idealized sets of superellipse parameter values according to some embodiments. In particular, metric Dx is determined for each image 1540, 1542, 1544 as a difference between the volume circumscribed by the associated parameter sets 1560, 1562, 1564 and the idealized versions of the parameter sets 1610, 1620, 1630. The image associated with the smallest value of D may be considered the most artifact-free image.

In addition to metrics 1580, FIG. 15 also shows metrics 1582, 1584 and 1586 determined by reconstruction evaluation component 1570. Metrics 1582 comprise the average value of parameter a for each of sets 1560, 1562 and 1564, metrics 1584 comprise the average value of parameter b for each of sets 1560, 1562 and 1564, and metrics 1586 comprise the average value of parameter n for each of sets 1560, 1562 and 1564. Embodiments may use any suitable combination of these metrics to evaluate the quality of reconstructed images 1540, 1542, 1544.

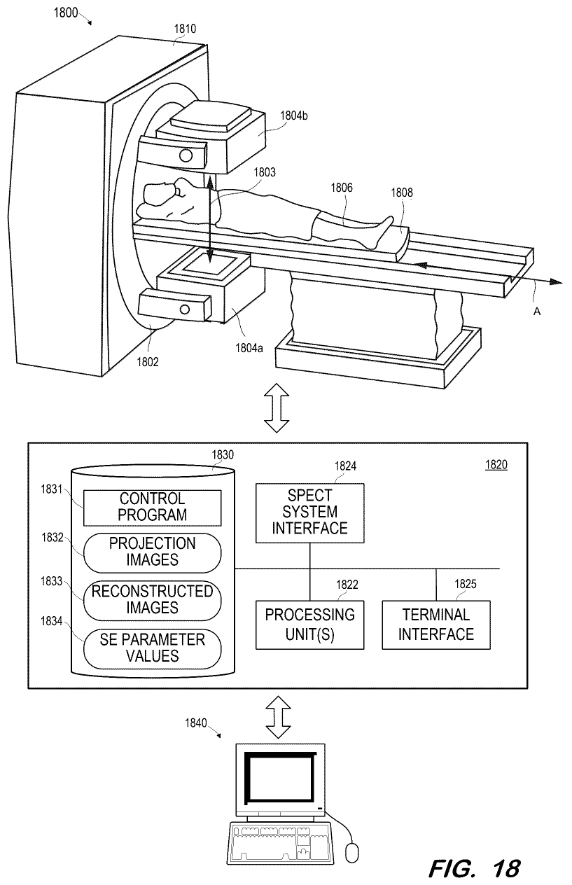

FIG. 18 illustrates imaging system 1800 for performing the processes described herein according to some embodiments. System 1800 is a SPECT imaging system as is known in the art, but embodiments are not limited thereto. As mentioned above, embodiments are not limited to an imaging system.

System 1800 includes housing 1810 for gantry 1802 to which two or more gamma cameras 1804a, 1804b are attached, although any number of gamma cameras can be used. A detector within each gamma camera detects gamma photons (i.e., emission data) emitted by a radioactive tracer injected into the body of patient 1806 lying on bed 1808. Bed 1808 is slidable along axis-of-motion A. At respective bed positions (i.e., imaging positions), a portion of the body of patient 1806 is positioned between gamma cameras 1804a, 1804b in order to capture emission data from that body portion from various projection angles.

Control system 1820 may comprise any general-purpose or dedicated computing system. Control system 1820 includes one or more processing units 1822 configured to execute executable program code to cause system 1820 to operate as described herein, and storage device 1830 for storing the program code. Storage device 1830 may comprise one or more fixed disks, solid-state random access memory, and/or removable media (e.g., a thumb drive) mounted in a corresponding interface (e.g., a USB port).

Storage device 1830 stores program code of control program 1831. One or more processing units 1822 may execute control program 1831 to, in conjunction with SPECT system interface 1824, control motors, servos, and encoders to cause gamma cameras 1804a, 1804b to rotate along gantry 1802 and to acquire two-dimensional emission data (i.e., projection images) 1832 at defined imaging positions during the rotation.

Control program 1831 may further be executed to reconstruct images 1833 from projection images 1832. Superellipse parameter values 1834 may be derived from reconstructed images 1833 as described herein.

Terminal 1840 may comprise a display device and an input device coupled to terminal interface 1825 of system 1820. Terminal 1840 may receive and display images 1833 and reference images 1834, as well as a user interface to assist comparisons therebetween. In some embodiments, terminal 1840 is a separate computing device such as, but not limited to, a desktop computer, a laptop computer, a tablet computer, and a smartphone.

Each component of imaging system 1800 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

What is claimed is:

1. A method comprising:
determining a first three-dimensional image;
for each of a plurality of first slices of the first three-dimensional image, fitting a model comprising a plurality of parameters to a first object in the first slice to determine values of each of the plurality of parameters;
determining values of each of the plurality of parameters for each of a plurality of second slices of a second three-dimensional image of a second object;
determining a difference between the values of each of the plurality of parameters determined for each of the plurality of first slices and the values of each of the plurality of parameters determined for each of the plurality of second slices; and
outputting an indication based on the difference.

2. The method of claim 1, wherein outputting an indication based on the difference comprises:
determining that the difference is greater than a threshold; and
in response to determining that the difference is greater than a threshold, outputting instructions to perform a device calibration.

3. The method of claim 2, wherein the first object and the second object each comprise a portion of a phantom.

4. The method of claim 2, further comprising determining calibration parameters for the device calibration based on the difference.

5. The method of claim 1, wherein outputting an indication based on the difference comprises:
determining that the difference is greater than a threshold; and
in response to determining that the difference is greater than a threshold, outputting an indication that motion artifacts of the first three-dimensional image exceed a threshold.

6. The method of claim 5, wherein the first object is a first left ventricle and the second object is a second left ventricle.

7. The method of claim 5, further comprising:
determining motion correction parameters based on difference; and
outputting the motion correction parameters.

8. The method of claim 1, wherein the first three-dimensional image was generated using first motion correction parameters and the second three-dimensional image was generated using second motion correction parameters,
wherein the indication is an indication of which of the first three-dimensional image and the second three-dimensional image exhibits a higher image quality.

9. The method of claim 8, wherein determining the difference comprises:
determining first representative values of each of the plurality of parameters determined for each of the plurality of first slices based on the values of each of the plurality of parameters determined for each of the plurality of first slices;
determining a first difference between the first representative values and the values of each of the plurality of parameters determined for each of the plurality of first slices;
determining second representative values of each of the plurality of parameters determined for each of the plurality of second slices based on the values of each of the plurality of parameters determined for each of the plurality of second slices;
determining a second difference between the second representative values and the values of each of the plurality of parameters determined for each of the plurality of second slices; and
determining whether the first three-dimensional image or the second three-dimensional image exhibits a higher image quality based on the first difference and the second difference.

10. The method of claim 1, wherein the model is a superellipse model and the parameters comprise a semi-major axis length, a semi-minor axis length and a squareness factor.

11. A system comprising:
an imaging system to acquire a first three-dimensional image;
a control system to:
for each of a plurality of first slices of the first three-dimensional image, fit a model comprising a plurality of parameters to a first object in the first slice to determine values of each of the plurality of parameters;
determine values of each of the plurality of parameters for each of a plurality of second slices of a second three-dimensional image of a second object;
determine a difference between the values of each of the plurality of parameters determined for each of the plurality of first slices and the values of each of the plurality of parameters determined for each of the plurality of second slices; and
output an indication based on the difference.

12. The system of claim 1, wherein outputting of the indication based on the difference comprises:
determination that the difference is greater than a threshold; and
in response to the determination that the difference is greater than a threshold, outputting of instructions to perform a device calibration.

13. The system of claim 12, wherein the first object and the second object each comprise a portion of a phantom.

14. The system of claim 10, wherein outputting of an indication based on the difference comprises:
determination that the difference is greater than a threshold; and
in response to the determination that the difference is greater than a threshold, outputting of an indication that motion artifacts of the first three-dimensional image exceed a threshold.

15. The system of claim 14, wherein the first object is a first left ventricle and the second object is a second left ventricle.

16. The system of claim 10, wherein the first three-dimensional image was generated using first motion correction parameters and the second three-dimensional image was generated using second motion correction parameters,
wherein the indication is an indication of which of the first three-dimensional image and the second three-dimensional image exhibits a higher image quality.

17. The system of claim 16, wherein determination of the difference comprises:
determination of first representative values of each of the plurality of parameters determined for each of the plurality of first slices based on the values of each of the plurality of parameters determined for each of the plurality of first slices;
determination of a first difference between the first representative values and the values of each of the plurality of parameters determined for each of the plurality of first slices;
determination of second representative values of each of the plurality of parameters determined for each of the plurality of second slices based on the values of each of the plurality of parameters determined for each of the plurality of second slices;
determination of a second difference between the second representative values and the values of each of the plurality of parameters determined for each of the plurality of second slices; and
determination of whether the first three-dimensional image or the second three-dimensional image exhibits a higher image quality based on the first difference and the second difference.

18. The system of claim 1, wherein the model is a superellipse model and the parameters comprise a semi-major axis length, a semi-minor axis length and a squareness factor.

19. One or more computer-readable media storing program code executable by one or more processing units of a computing system to cause the computing system to perform operations comprising:
determining a first three-dimensional image;
for each of a plurality of first slices of the first three-dimensional image, fitting a model comprising a plurality of parameters to a first object in the first slice to determine values of each of the plurality of parameters;
determining values of each of the plurality of parameters for each of a plurality of second slices of a second three-dimensional image of a second object;
determining a difference between the values of each of the plurality of parameters determined for each of the plurality of first slices and the values of each of the plurality of parameters determined for each of the plurality of second slices; and
outputting an indication based on the difference.

20. The one or more computer-readable media of claim 19, wherein the first three-dimensional image was generated using first motion correction parameters and the second three-dimensional image was generated using second motion correction parameters,
wherein the indication is an indication of which of the first three-dimensional image and the second three-dimensional image exhibits a higher image quality, and
wherein determining the difference comprises:
determining first representative values of each of the plurality of parameters determined for each of the plurality of first slices based on the values of each of the plurality of parameters determined for each of the plurality of first slices;
determining a first difference between the first representative values and the values of each of the plurality of parameters determined for each of the plurality of first slices;
determining second representative values of each of the plurality of parameters determined for each of the plurality of second slices based on the values of each of the plurality of parameters determined for each of the plurality of second slices;
determining a second difference between the second representative values and the values of each of the plurality of parameters determined for each of the plurality of second slices; and
determining whether the first three-dimensional image or the second three-dimensional image exhibits a higher image quality based on the first difference and the second difference.
